(19) United States
(12) Patent Application Publication
(10) Pub. No.: US 2022/0125029 A1
(43) Pub. Date: Apr. 28, 2022

(54) Insect and Creature Monitoring System

(71) Applicant: Ingenueering, LLC, Montgomery, TX (US)

(72) Inventor: Mario Chapa, Montgomery, TX (US)

(21) Appl. No.: 17/429,044

(22) PCT Filed: Feb. 5, 2020
(86) PCT No.: PCT/US20/16840

§ 371 Date: Aug. 6, 2021

(51) Int. Cl. A01M 1/02; A01M 1/04; A01M 1/14; G06Q 50/02

(52) U.S. Cl. CPC A01M 1/026; G06Q 50/02; A01M 1/145; A01M 1/04

(57) ABSTRACT

The present application describes a pollinator species detection and monitoring device and system that utilizes specifically positioned and spaced receptacles for determining and conveying the numbers, health and movements of pollinator species ranging from real-time to intermittent transmittance of detected information for an area, region, areas or regions, singularly and in the aggregate. The detected information is collected and recorded via a pair of closely related sensors, residing at 180 degrees from one another, wherein pollinator species' entrance, exit, number, gender and activity are sensed, analyzed algorithmically and stored locally for eventual transmittance from a transmitting device to an external computer or mobile device for data observance, collection, storage and analysis. Detected information may be species specific or across a plurality of species.

### CROSS-REFERENCE TO RELATED APPLICATIONS

PCT International Patent Application No. PCT/US19/16840 filed Feb. 6, 2019

### FEDERALLY SPONSORED RESEARCH AND DEVELOPMENT: NOT APPLICABLE

### Incorporation by Reference

Not Applicable

### BACKGROUND

### Field of the Invention

The present invention relates to a device and method utilized to detect the numbers, health and movements of pollinator populations and various pests, generally, and to individual monitoring devices, placed at regularly occurring spatial intervals, that are used to track, map, count and record various pollinating species, specifically. Such information is then detected and transmitted as data to an analytical device ranging from programmed or induced real time to daily, weekly, monthly and regular and irregular transmissions.

### Description of the Related Art

Terrestrial ecosystems are undergoing changes that have historically occurred over centuries to millennia, but are now transforming the geographic condition of earth's surface at rates that are readily observable, now from decades to years. At an ever-accelerating rate, the untoward effects of global greenhouse gases (e.g. carbon dioxide, nitrous oxide and methane) can be observed in rising sea levels, changing precipitation patterns and the increased proliferation of deserts and similarly arid climates. This gradual heating of the earth's climate system, together with dwindling pollinator habitats and the ubiquitous use of pesticides, stands as one of the primary consequences of a persistent consumption and consumerism that to date has only hastened pollinator number's decline in rapidity and accentuated their deleterious effects. It is this decline in pollinator species that stands to adversely affect not only pollinators themselves but overall human survival going forward.

Pollinators (including insects, vertebrates and mammals) are critical to the proper reproduction of flowering plants constituting a wide variety of consumable horticultural crops which represent a staple of human food consumption worldwide. Indeed nearly 90% of flowering plants have at least some dependence upon pollen transfer by insects and animals' and some 35% of the world's global crop production is equally pollinator-dependent (wherein bees constitute a large portion of the total pollinator population).² Ironically, as human populations grow, agricultural intensive methods (dependent upon a handful of highly productive crops) are employed to support this burgeoning population expansion. Increased food demand results in utilization of larger and larger areas of land for agricultural purposes wherein land use encroaches upon the habitat of pollinators, changes the ecological systems upon which these very organisms rely, creates significant losses in biodiversity among employed staple crops and ultimately operates counter the long-term survival of the human species. Manifestly, it is not only the effects on the land that is used for increased crop production but also the untoward effects on terrestrial and aquatic resources (i.e. those agricultural resources that are not directly utilized in crop production) that nonetheless remain susceptible to residual effects and exhibit secondary damages that even further exacerbate this threat.

The inherent dichotomy of increased food demand of an expanding population with a decreased pollinator supply creates a vicious cycle of ever-decreasing returns. The intensive, undiversified agricultural expansion supported by increasingly devastating (lethal and sublethal) agrochemical use³, decreases in indigenous weedy flowing species that co-support pollinators, reduction in habitat acreage and GHG hastened climate change (resulting in adaptive responses and altered seasonal activities due to fluctuations in global temperatures) all contribute to a devolving ability to meet the nutritional needs of a world that is advancing in human numbers at an exponential rate. Sadly, as agricultural-based, population driven demands expand, more land is utilized for planting which encroaches on the pollinator's natural habitat and further burdens a smaller and smaller workforce. Namely studies have found that more than 40% of insect pollinators globally are highly threatened, up to and including bees native to the United States.⁴ And, not only do these crops aid in human sustenance but also in providing employment and livelihood to millions worldwide.

Pollination, itself, is defined as the transference of pollen or, in other words, the movement of male genetic material from the male reproductive structure of one plant to the female reproductive structure of another plant which can be via wind, animal and insect vectors or through a self-pollinating process. A majority of plants rely upon pollinators in the form of insect and animal vectors that may include any number of winged insects (bees, flies, butterflies, wasps and moths), winged mammals (birds and bats) or other mammals (primates and rodents). Chief among all pollinators though is the bee.

Yet, it is not only the quantity of human consumable crops that undergo declines in the above situations, it is also the quality. While consumable crop harvests may vary extensively according to crop and location, in terms of yield quality, a combination of overall declines in pollinator species' biodiversity, married with a slant toward highly productive crop species, only serves to affect the diversity within single crops. Manifestly, a varied selection among one group of pollinators can have a tremendous effect even where even a large number of monochromatic species can result in less effect and less stable crop production.¹

Undeniably, decreases in GHG emissions and resultant global temperature decrease have the greatest potential for normalizing pollinator seasonal activity in the macro, but increasingly conscious agricultural management systems (e.g. uncultivated vegetation areas, grassland management, diversified crop rotation and restoration of natural habitats), conscientious (reduced) agrichemical use and reduced pollution and decreasing invasive alien species can have appreciable in the micro. Yet, with all of these remunerative measures, it is of the most vital importance to study, observe and understand pollinator species in terms of number, location and patterns as well as the ambient conditions within which they function (e.g. light intensity, wind velocity, temperature, rainfall and relative humidity).

Current methods for counting and tracking pollinator species consists of a receptacle, usually in the form of a box, containing a pheromone attractive to the desired species. Typically, the box has an opening that allows for both pheromone egress and species entrance. Commonly, the box contains, as well, a “flypaper” to trap the designated species. After a set period of time, a technician must retrieve the flypaper in order to manually count the number of species (e.g. insects) individually. This method of counting takes time and continuous human intervention in the counting process as a person must travel from box to box and manually count insects and manually input data into a database. This process is not only arduous, physically demanding and labor intensive, it is also slow and prone to human error. What is more the periodic inspection of these pollinator species suffers from sporadic and inconsistent monitoring and assessment of each species.

While strides have been made to overcome the inadequacies of monitoring pollinators, it remains evident that considerable failings remain in the field. It is the goal of the present invention to remedy these shortcomings as to allow better monitoring and management of these species and to potentiate a system of improved understanding of the delicate operations and functions within and between pollinator, an non-pollinator, species.

While inventor has set forth the best mode or modes contemplated of carrying out the invention known to inventor such to enable a person skilled in the art to practice the present invention, the preferred embodiments are, however, not intended to be limiting, but, on the contrary, are included in a non-limiting sense apt to alterations and modifications within the scope and spirit of the disclosure and appended claims.

### SUMMARY OF THE INVENTION

The present invention consists of three main parts: a bait, a box and a microcontroller unit that is further subdivided into sensors, memory, power and data collection and transmittance modules. The bait consists of an interchangeable pheromone or any other suitable bait (i.e. food, honey or sugar) or decoys such as lights or sounds (or species specific pray animals) that are emitted from inside a box containing one or more gates to the internalized bait. As the odor or light of the bait is expelled out of the box through the gates, either by natural means or facilitation (e.g. via a fan in the case of pheromones), the odor, sound or light attracts the desired pollinator to enter the box through said gates. The species size-adjustable gates are designed with an array of appropriately-sized detection sensors that monitor the number of designated pollinators (or non-pollinator species) that enter and leave the box. What is more, these sensors can be calibrated to further track the speed, direction and, in some cases, the breed and gender, of a particular species.

In a preferred embodiment, a pair of two photo-interrupter sensors are placed in the interior of each gate, 180 degrees apart, wherein the photo-interrupter itself is configured as (1) an optical emitter and (2) an optical receptor/detector that further incorporates an electrical logic-level output for data transmittance. When the optical beam (e.g. an infrared LED) is blocked by an object, the logic output state switches to signal the interruption of the signal (beam) and thus provides data showing a body entrance or exit. Additionally, when used in tandem, a pair of photo-interrupter sensors at a known distance (6 mm in the present device for bee monitoring) from one another can be used, incorporating sequence and timing, to determine not only entrance and exit, but also speed and size (length) where triggering the primary gate first and the secondary gate second signifies entrance, triggering the secondary gate first and the primary gate second signifies exit, and the timing between signal interruption, either upon entrance or exit, with a known distance (D), allows for a determination of length (and therefore size) as a function of time (T) where D=S (speed)×T (time). What is more, knowing the relative size difference between males and females in a species population, it may also be known what gender is entering or exiting through each gate pair (e.g. the relative larger size of a queen bee is known in relation to a drone bee's size in relation to a worker bee's size). In terms of configuration, the photo (light) emitting sensor on one gate interior ‘leg’ is made to generate and transmit light to a photo (light) receiving sensor on the opposite, opposing inward ‘leg’ of the same primary (and exteriorly positioned) gate. Reciprocally, a secondary gate, that is positioned between the primary gate and the exterior of the present device, is designed to exhibit a photo emitting sensor that projects light in the opposite direction, in relation to the primary gate, wherein, on the interior portion on each gate ‘leg’, there is a receiving sensor on each same side exhibiting an emitting sensor and an emitting sensor on each same side exhibiting a receiving sensor. Concisely, where one sensor transmits light generally from left to right, the second sensor transmits light from right to left, and vice versa. This 180-degree orientation is crucial in that emissions and reception exclude the possibility of reception of photo interference by the next adjacent gate photo emitter to the matching photo receptor. And while photo-interrupter sensors are utilized specifically in the above preferred embodiment, it is within the contemplation of the applicant to use various types of sensors including, but not limited to, photogate reflective light sensors, hall effect sensors or electromagnetic sensors.

In another preferred embodiment a pollinator species may include winged insects (bees, flies, butterflies, wasps, hornets and moths), winged mammals (birds and bats) or other mammals (primates and rodents) and other non-pollinator species having interspecies and/or untoward environmental impacts (i.e. beetles and locusts) with reference to long-term human sustenance. By designating each species individually, the photo gates, and photo sensor arrays, may be adjusted in size, accordingly, as to accommodate differing species' sizes.

In another preferred embodiment a sticky paper in the form of a flypaper could be used to act as an alternative means of enhancing, verifying and/or validating the collected and accumulated data.

In yet another preferred embodiment the present invention may also contain a plurality of sensors that, in addition to measuring pollinator species, are capable of measuring light intensity, wind velocity, temperature, relative humidity, soil humidity, soil pH and the like. Additionally, accelerometers, inclinometers and weight and rain gauges may be employed to measure ambient conditions both inside of and outside of the device in furtherance of understanding the overall health of a species (e.g. where weight and internal and external temperature play a vital role in the determining the overall health of a bee hive). In addition to accelerometers, mechanical switches and triggers, sound and motion detectors, audio and video recording devices and infrared detectors may equally be employed.

In one preferred embodiment, in terms of configuration, the set of gates' number, size and shape can be adjusted to accommodate the particular species that is being monitored. The box itself can be made of a material that is transparent, opaque or a combination thereof. Transparent materials can be used in conjunction with a decoy or bait that resembles a particular type of bird or insect in order to attract a particular species. Water, food, light, smell, sound, singly or in combination, can be used as a lure to attract particular species

In yet another preferred embodiment, gates can be fitted with structures that look like specific flowers, flowering plants or fruits to attract certain creatures who express a specific proclivity toward such structures (e.g. hummingbirds or bats). The flower, flowering plants or fruits can equally be fitted with sensors to tabulate species contact with each structure. Moreover, cameras and microphones can also be employed to capture images and sounds of creatures specific to the designated flowers, flowering plants or fruit.

In another preferred embodiment, collected data from the microcontroller is stored in the device's internal memory and may be transmitted at programmable or user-initiated intervals to a “cloud” storage via radio frequency, Wi-Fi, cellular network or any other form of suitable communication. If real time data is required, the transmission frequency can be very short, such as a few minutes. If power, though, is a concern, longer intervals between transmission may be utilized (e.g. once daily, once weekly, once monthly or yearly). An onboard firmware with a programmable “over the air” application would allow the user a “programmable” or “reprogrammable” transmittance interval or a “two-way” communication avenue allowing adjustment of transmission frequencies in addition to other parameters (i.e. choice of transmission means and retention of collected data) where the user can override the transmission frequency to allow for a selectable transmission interval or to push data transference in real-time.

In another embodiment, a cloud application and data base stores and aggregates collected data from multiple devices and creates population maps, collectively, to track and predict population mechanics and movements of a single pollinator species or a plurality of pollinator or non-pollinator species. Insects such as pests (e.g. beetles and locusts), pollinators (e.g. bees, butterflies, hornets, wasps, ants, moths and flies) may be tracked. Other animals such as humming birds, bats, squirrels or similar mammals can also be counted and tracked.

In yet another embodiment, light interruption and timing of said light interruption between a pair of photo-interrupter sensors can be used to determine, in the case of bees, a hive's overall health by determining the entrance and exit of a particular population based on species gender. Namely, a bees gender may be determined by equating relative size to photo sensor interruption timing and sequence where the time to traverse a distance by a body is calculated from interruption of said first photo interrupter to discontinuation of disruption of said second photo interrupter—where it is known that, in the case of bees, male drones have a body size greater than that of worker bees (albeit smaller than that of the queen) and shifts in population and population gender composure can signify patterns and events known to affect total hive health.

In another preferred embodiment, the device may be used as part of a collection of devices, of species-specific size, may be placed on a pole at differing heights, angles and positions (with different baits or pheromones) to attract different kinds of insects and creatures within the same geographic location or across several geographic locations. Further, the device can be placed (or hung) in organic structures (e.g. trees), existing structure such as buildings, homes, bridges and/or in the ground or under water. The collection of devices may be placed at regular or irregular intervals to cover vast territories or specific areas of interest to determine how insect or animal populations move or emerge from the ground across an area, region, an entire country or across a body of water.

In one embodiment the device can be powered by a solar panel, a battery, a wind turbine or a combination of a solar panel, a battery, wind turbine or connected to another power source, renewable or non-renewable.

In another embodiment different types of lids and doors may be installed to access the lures, baits, sensors, batteries, memory devices and fly paper as deemed necessary for installation, replacement and maintenance of each component.

In another preferred embodiment a display may be affixed to the interior or exterior of the device to provide information visually to represent information such as battery power or sensor data.

### BRIEF DESCRIPTION OF THE DRAWINGS

While the novel features and method of use of the application are set forth above, the application itself, as well as a preferred mode of use, and advantages thereof, will best be understood by referencing to the following detailed description when read in conjunction with the accompanying drawings in view of the appended claims, wherein:

FIG. 1 depicts the present invention as it relates to detecting, tracking, counting and recording pollinator species bees.

FIG. 2 shows photo interrupter sensors as employed on the plurality of entrance and exit gates of the present invention.

FIG. 3 depicts the interchangeable gate array for accommodation of several detectable species.

FIG. 4 depicts the 180-degree orientation of a set of sensor pairs in pollinator detection.

FIG. 5 illustrates T₀ to T₃ for the detection of species entrance and exit as well as species speed, length and size.

FIG. 6 shows a diagrammatic representation of a sensor set, microcontroller, memory, battery, transmittance module, cloud storage and user reception used in data collection, storage and conveyance.

FIG. 7 illustrates a slack protocol of species entry and exit.

FIG. 8 is a strict protocol algorithm for species detection and entrance and exit conformation.

FIG. 9 shows elevated placement of the present device.

FIG. 10 shows an alternative position of device placement.

FIG. 11 is an expanded view of the alternative device position of FIG. 10.

FIG. 12 is the externally mounted unit that is made to house the microcontroller, battery, sensor connectors, photo gate connectors, communication and transmission connectors and connectors and power connectors internally and entrance and exit gates externally.

FIG. 13 is an interior view of the present device.

FIG. 14 represents an expanded internalized view of the present invention.

FIG. 15 shows the lure, decoy or bait platform that is used as an attractant in the present invention.

And while the invention itself and method of use are amendable to various modifications and alternative configurations, specific embodiments thereof have been shown by way of example in the drawings and are herein described in adequate detail to teach those having skill in the art how to make and practice the same. It should, however, be understood that the above description and preferred embodiments disclosed, are not intended to limit the invention to the particular embodiment disclosed, but on the contrary, the invention disclosure is intended to cover all modifications, alternatives and equivalents falling within the spirit and scope of the invention as defined within the claim's broadest reasonable interpretation consistent with the specification.

### DETAILED DESCRIPTION OF THE PREFERRED EMBODIMENTS

A detailed description of the preferred embodiments of the invention is disclosed and described below. Yet, each and every possible feature, within the limits of the specification, are not disclosed as various permutations are postulated to be in the purview and contemplation of those having skill in the art. It is therefore possible for those having skill in the art to practice the disclosed invention while observing that certain placement and spatial arrangements are relative and capable of being arranged and rearranged at various points about the present invention that nonetheless accomplishes the correction of one or more of the infirmities as outlined and discussed above in the field of both monitoring and management of pollinator species. Patently, the size and shape of certain features may be expanded or narrowed to accommodate each individual species and may be customizable to suit each species individually.

Equally, it should be observed that the present invention can be understood, in terms of both structure and function, from the accompanying disclosure and claims taken in context with the associated drawings. And whereas the present invention and method of use are capable of several different embodiments, which can be arranged and rearranged into several configurations, which allows for mixing and matching of features and components, each may exhibit accompanying interchangeable functionalities, which may be species specific, without departing from the scope and spirit of the present application as shown and described.

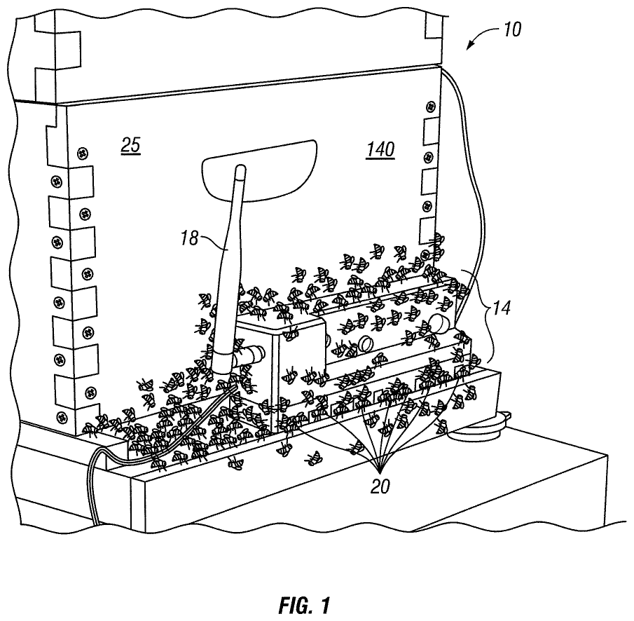

As detailed in FIG. 1, the representation of the present device 10 including the collection and transmittance of data collected with relation to the a specific, selected pollinator species, in this case bees, focusing on the externally mounted unit 14 that is comprised, generally, of a plurality of gates 20, an internalized microcontroller (not depicted) and a transmittance device 18 that is mounted externally to an enclosed space in the form of an enclosure 25 (e.g. a beehive). In addition, transmittance device 18 may also work as a receiving unit for programming and reprogramming of on-board firmware where data type and data transfer frequency may be modified by the user, in a retrograde fashion, from a user-cloud platform. It is the function of gates 20 to allow for entrance and exit of a species of bees, recordation and collection of the movements and speed of this species and transmittance of that collected data to a conveyance module which is ultimately received by a cloud storage for ultimate re-transmittance to a receiving device (e.g. computer or mobile device) for user data observation, collection, storage and analysis.

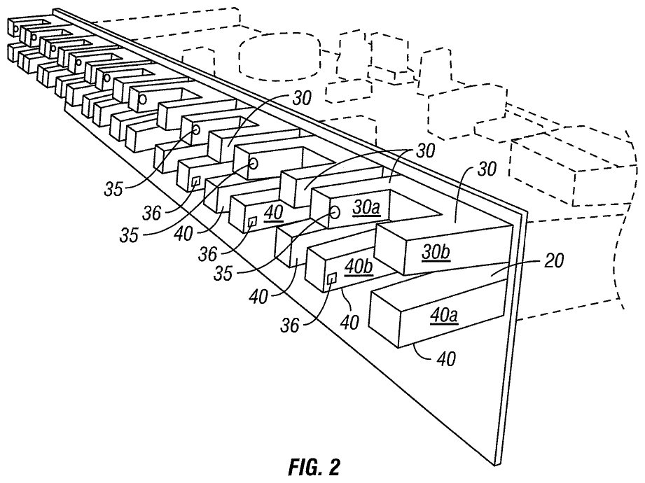

FIG. 2 provides a prospective view of a plurality of individual gates 20 wherein the first sensing unit 30 is superior to the second sensing unit 40 as depicted, but would find gates 20 in their installed orientation wherein the first sensing unit 30 is exterior to the second sensing unit 40 in the actual device 10 and the second sensing unit 40 is positioned between the first sensing unit 30 and the primary enclosure 140 exterior. Further, signal emitting unit 35 is visible projecting a emitted signal from the interior ‘leg’ of the first sensing unit 30a to a receiving portion of the interior ‘leg’ of the first (outer) sensing unit 30b (not shown) and the signal detection sensor 36 of second (inner) sensing unit 40b (which is a reciprocal of the emitting unit 35) is made to receive a signal from the signal emitting unit 35 from the interior of second (inner) sensing unit 40b (not shown). Therefore, the emitted signals from 30a and 40a are oriented 180 degrees from one another as to obviate any cross reactivity and interference that would result from light being emitted from the interior of 30a affecting the signal reception 40a and the interior signal emittance of 30b affecting the signal reception of 40b, in a same sided orientation, where emitting sensors 35 placed on the interiors of parallel sensing units are detected by reciprocal receiving units 36 and each emitting unit 35 and reciprocal receiving unit 36, deposed in the lower third of each sensing unit's 30, 40 inner portions and each first sensing unit 30 is a set, predetermined and calculable distance from each second sensing unit for the computation of entrance, exist speed and size/gender of a species.

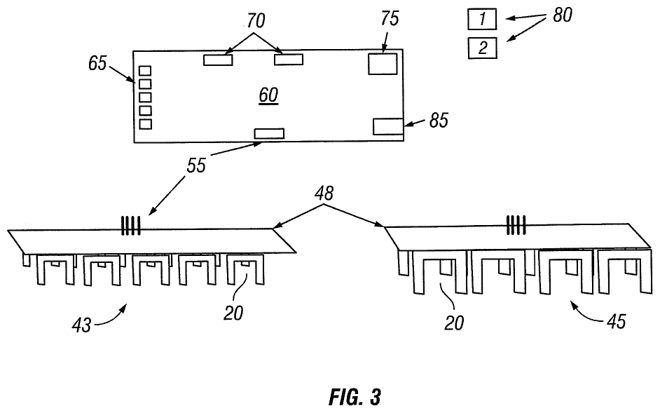

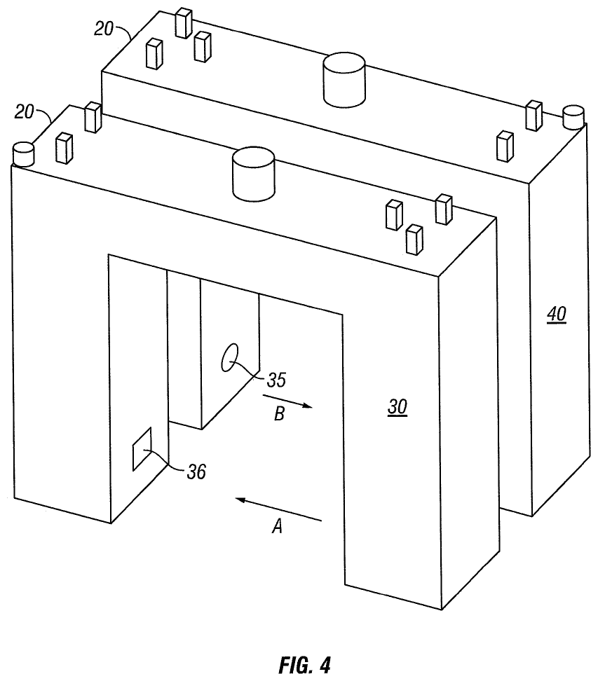

As is described above, the present invention is modifiable and adaptable to accommodate any number of pollinator (and non-pollinator) species of various and varying sizes. It is therefore within the contemplation of inventor to allow for gates 20 that are selected and replaceable, gate set 45 being larger than gate set 43, based on the particular species that is to be observed. FIG. 3 depicts a series of gates 20, in the form of an interchangeable photo gate array 48 that is connected to photo gate connectors 55 which relay information to the microcontroller 60. As provided in greater detail, FIG. 4 depicts one set of sensing devices exhibiting a 180-degree emission-reception relationship where the first sensing unit 30 encompasses internalized sensor emission from right (emission) to left (reception) and inner gate 40 encompasses internalized sensor emission from left (emission) to right (reception) exhibited where first (outer) sensing unit 30 evidences receiving sensor 36 and second (inner) sensing unit 40 evidences emission unit 35, the direction of which are shown, via representational arrows A and B, respectively. As well as the photo gate connectors 55, various other detectors, in the form of sensor connectors 65 may as well be utilized for various ambient sensors 90 (i.e. accelerometers, inclinometers and weight and rain gauges that may be employed to measure ambient conditions both inside of and outside of the device) which may also include supplemental additional connectors. Once recorded and collected, the data compiled in the microcontroller 60 is then transmitted, via a transmittance device 18 (FIG. 1) integrated through communications module connectors 75 wherein communication module connectors 80 may also be interchangeable to accommodate radio frequency, Wi-Fi, cellular network or any other suitable form of communication as conditions and area dictate. Further, a power connector 85 is integrated into the microcontroller 60 as to allow for battery or solar power.

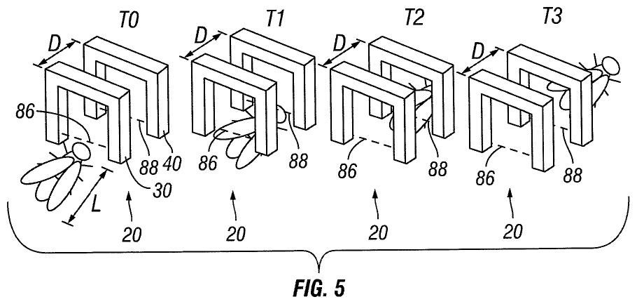

Expounding upon the relationship between each inner and outer sensing unit 30 and 40, and a defined and quantified distance (D), the present device utilizes time (T) via T₀, T₁, T₂ and T₃, as described and shown in FIG. 5, to determine not only the binomial entrance and exit of an individual pollinator through beam interruption but also, where D is known, the length (L) (i.e. insect size) of each individual entering or exiting the present invention through the device gates where length L relates to speed S and differences in time T. Succinctly, L is calculable where T and S are supplied and length functions to determine the gender of an individual (e.g. length/size is determinable where L=S×T). Specifically, a pollinator or non-pollinator species breaks the plane of the first beam 86 of first sensing unit 30 at T₀. The pollinator then breaks the plane of the second beam 88 of the second sensing unit 40 at T₁ where Speed (S) is calculable as S=D/(T₁−T₀). At T₂ the first beam 86 is once again continuous/received (i.e. restored) to record T₂. Once the second beam 88 is restored, length, and therefore gender may be known through the equation:

L≈S*(T₃−T₂)

Specifically, where the pollinator species is a bee, it is known that the queen is larger than drones (males) which are considerably larger than worker (females) where a greater L would equate to a larger insect and would allow the device user to determine gender where queen>drone>workers. With the corresponding ability to determine gender, it is possible, together with accompanying sensor information, such as internal hive temperature, hive health, humidity, hive cycle and season, to better understand the habits of queen bees with relation to worker bees as opposed to drone bees for example.

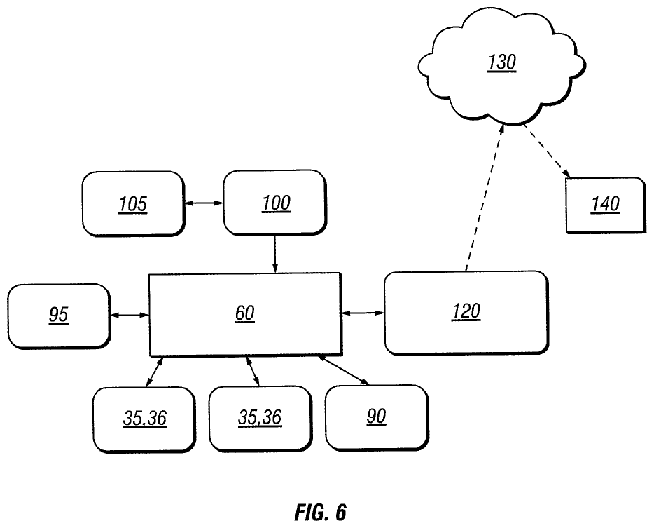

As represented diagrammatically in FIG. 6, microcontroller 60 is connected to a series of sensors that could include gate sensors 35, 36 or ambient sensors 90 (including accelerometers, inclinometers and weight and rain gauges may be employed to measure ambient conditions both inside of and outside of the device) coupled with a memory storage 95. Supplying power to the microcontroller 60 can be accomplished through a conventional battery 100 or a battery 100 that is powered by a polar panel 105. Once data is processed via the microcontroller 60, that data is either transmitted directly to cloud storage 130 via an interchangeable communication module 120 (e.g. a wi-fi or cellular transmission) that is ultimately communicated to a device user 140 for observation, measurement and analysis. This information may have intervals that are predetermined, programmable or requestable to be “pushed” to the user.

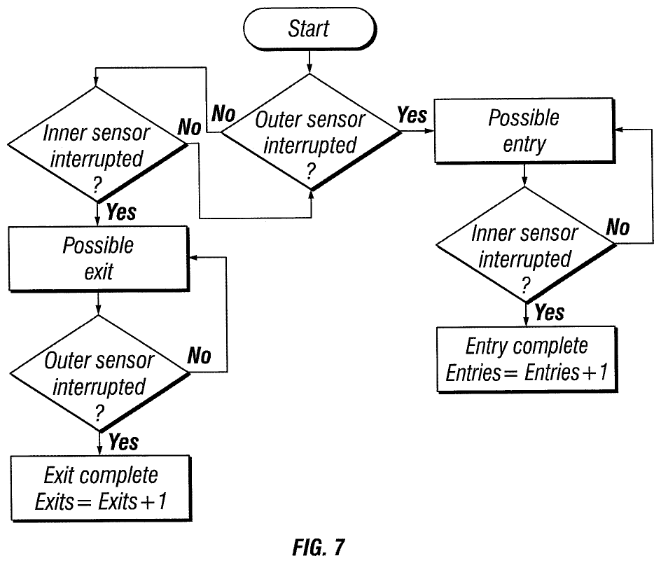

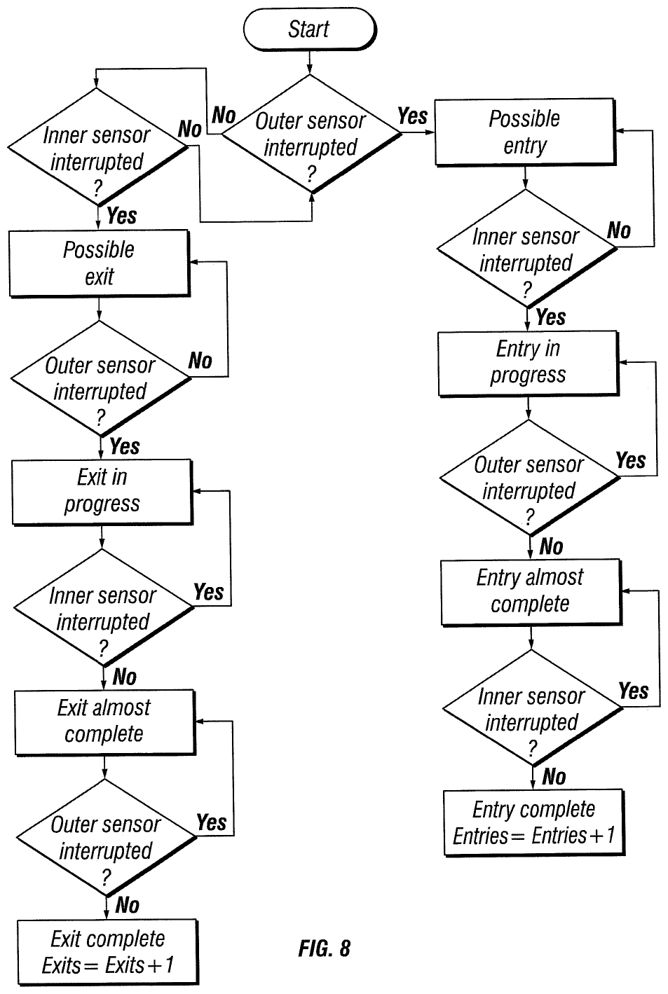

FIGS. 7 and 8 represent the algorithmic slack protocol and strict protocol, respectively, for determining the interpreting a particular species position upon sensor interruption and the observance that a species has either entered or exited the device based on the sequence of interruption and the continuation or discontinuation of sensor interruption as a function of time (T). As can be seen the slack protocol is less involved and can be relied upon to detect entrance and exits, explicitly, whereas the strict protocol allows for a nuanced and more sensitive collection of data that ultimately lends itself to not only entrance and exit but also a temporal designation that is required to determine species speed (S above) and ultimately gender where length is determinate of species gender.

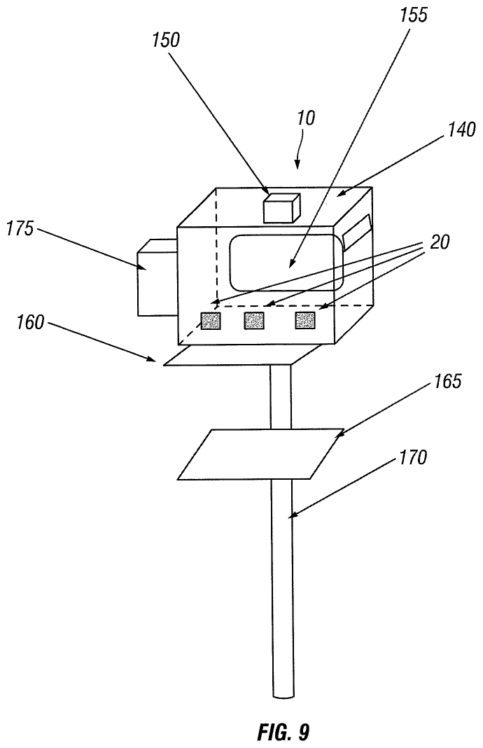

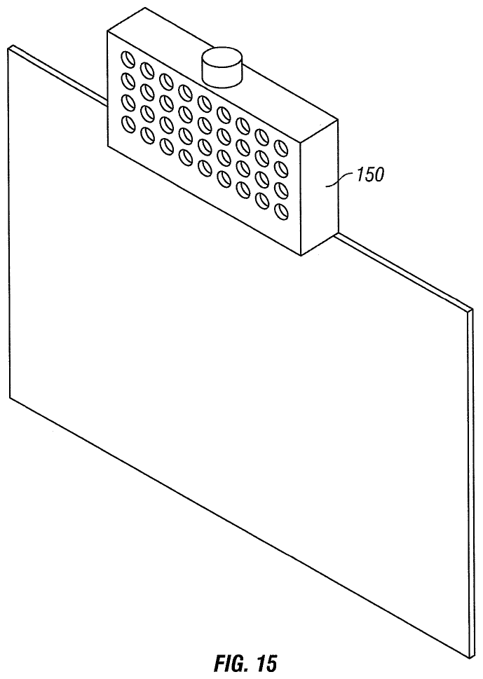

FIGS. 9-15 provide a diagrammatic representation of the present device 10. As shown in FIG. 9, this device 10 may consist of a primary enclosure 140 that may consist of a box and lid configuration positioned atop a pole 170, or similar structure, exhibiting one to a plurality of gates 20 (that may be disguised as species specific fruit or flowers) serving as the sole point of entrance and exit (although multiple access points have been contemplated with similar gating systems). The primary enclosure 140 may as well contain a lure, decoy or bait compartment 150 to induce the collection of data or fly paper 155 to substantiate or validate data collection. In addition, the device 10 that is the present invention can be made to harbor a landing pad 160 and/or a solar panel 165 in attempts to facilitate creature landing and extended battery use, respectively. What is more FIG. 9 is illustrated with an external compartment 175 that may be made to house a microcontroller 60, ambient sensors 90, memory 95, a battery 100 and a communication module 120.

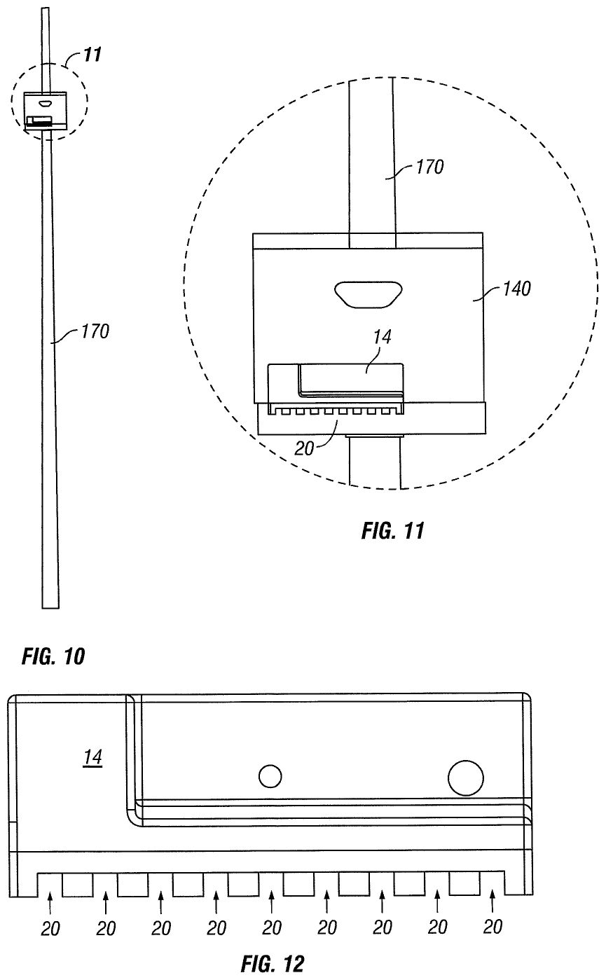

FIG. 10 depicts a device 10 with a positional relationship between the primary enclosure 140 and an adjustable height at some location about a pole 170 upon which it rests. The enlarged view of device 10, as shown in FIG. 11, evidences a single, external housing unit 14 consisting of a microcontroller 60, memory 95, battery 100 and various connectors (see FIG. 3) residing on the exterior of the device 10 that is shown as a primary enclosure 140 consisting of a box structure wherein gates 20 (as provided in magnified FIG. 12) are in close proximity to data collection and data relay circuitry.

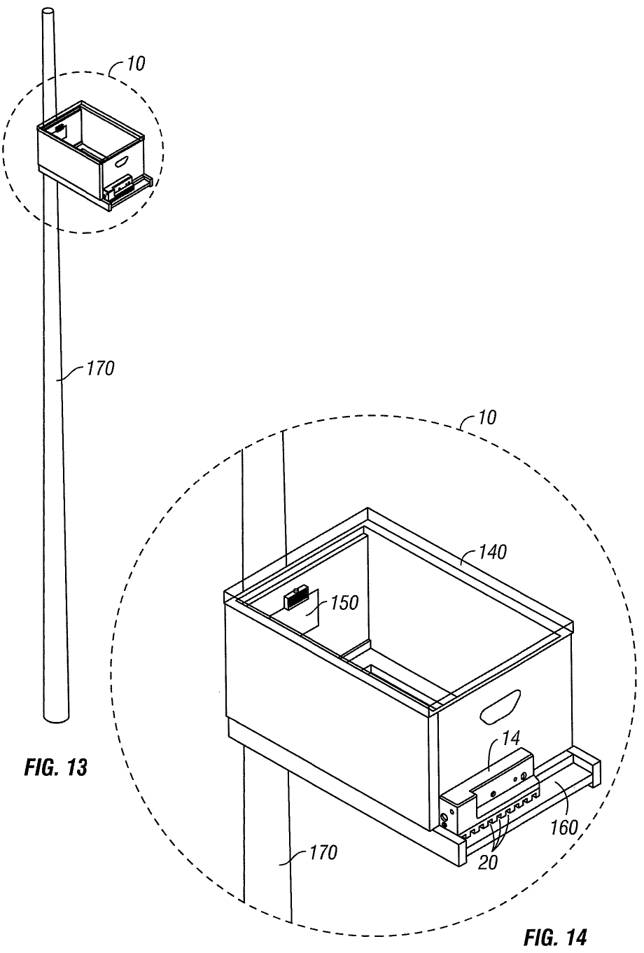

FIGS. 13-15 exhibit a perspective view of the internal compartment of the device 10 positioned at a point below the pole's apex wherein a removable decoy or bait compartment 150 is depicted in an internal area opposite the gated entrance of the primary enclosure 140. Although the decoy or bait compartment 150 is depicted as a passive diffusion, it is contemplated by the inventor to also encompass an active diffusion that may facilitate the emission of scents (in the form of food or pheromone scents) through induced air movement throughout the primary closure and through the externally residing gates 20. It is further contemplated that the decoy or bait compartment may be positioned in other areas within the device 10 itself and/or externally about the device 10.

The present invention evidences many advantages over the prior art including at least the following: (1) the ability to autonomously measure and track various species—both pollinator and non-pollinators—with one modifiable device, (2) the ability to measure the entrance, exit, speed, length and gender of a species through a pair of closely related sensor gates as a function of time (T), (3) the capability to transmit data in real time, or in a time delayed fashion, via a single programmable unit, (4) the ability to accept programming instructions with which to adjust the content or timing of transmitted data by the user, and (5) the capacity to aggregate data collected from several devices collecting data across an individual species population or across several species populations in a given area, region or across several areas or regions.

The particular embodiments disclosed are merely illustrative, which may be apparent to those having skill in the art that may be modified in diverse but equivalent manners. It is therefore contemplated that these particular embodiments may be altered and modified and that all such alterations are considered within the scope and spirit of the present application. And while these illustrations are of a limited number set, it is clear that the invention itself is mutable to any number of arrangements, configurations and modifications without departing from the invention's spirit thereof.
1. Intergovernmental Science-Policy Platform on Biodiversity and Ecosystem Services (IPBES). 2016. Summary for policymakers of the assessment report of the Intergovernmental Science-Policy Platform on Biodiversity and Ecosystem Services on pollinators, pollination and food production. Potts, S. G., et al. (editors) Available from: https://www.actu-environnement.com/media/pdf/news-26331-synthese-ipbes-decideurs-pollinisateurs.pdf (accessed Oct. 29, 2018)
2. Biodiversity for a World Without Hunger. Food and Agriculture Organization of the United Nations.
3. Rundlof et al., 2015. Seed coating with a neonicotinoid insecticide negatively affects wild bees. Nature 521: 77-80 4 doi:10.1038/nature14420
4. Pollinators in peril: A Systemic Status Review of North American and Hawaiian Native Bees. Kelsy Kopec & Lori Ann Burd. Center for Biological Diversity. February 2017

What is claimed is:

1. A pollinator species detection device for measuring and collecting data of a species, comprising:
an external ambient sensor, internal ambient sensor or both;
an enclosure functioning as a pollinator species collection device with a single access point for entrance and exit;
a gate or plurality of gates for pollinator species entrance and exit wherein each gate has a first sensing unit and a second sensing unit;
said first sensing unit and a second sensing unit operating in tandem;
said first sensing unit positioned external to a pollinator collection device;
said second sensing unit positioned external to a pollinator collection device between said first sensing unit and said pollinator species collection device;
said first sensing unit exhibiting a movement detection means wherein a photo beam emitting portion and a photo beam receiving portion are interiorly positioned at 180 degrees from the one another;
said second sensing unit having a movement detection means wherein a photo beam emitter and a photo beam detector are positioned interiorly at 180 degrees from the one another;
said second sensing unit exhibiting a photo beam emitter that is 180 degrees from said first sensing unit's photo beam emitter and said second sensing unit exhibiting a photo beam receiving detector that is 180 degrees from said first sensing unit's photo beam receiving detector;
a microcontroller system comprising a memory module, a data collection module, a transmitter module and a power source for receipt and transmittance of sensor data.

2. The pollinator species detection device of claim 1, wherein said sensing unit may be a photo interrupter, reflected light sensor, hall effect sensor, electromagnetic sensor accelerometers, mechanical switches or triggers, sound and motion detectors or infrared detectors.

3. The pollinator species detection device of claim 1, wherein the detection means is a pair of photo interrupter sensors, utilized in tandem, at a specific and determinable distance from one another wherein the detection of a species is dependent upon light interruption, the sequence of light interruption and the timing of light interruption to determine the entrance, exit and speed of a species.

4. The pollinator species detection device of claim 3, wherein said light interruption and said timing of said light interruption between said pair of photo interrupter sensors determines species gender by equating relative size to photo sensor interruption and timing where the time to traverse a distance by a body is calculated from interruption of said first photo interrupter to discontinuation of disruption of said second photo interrupter or interruption of said second interrupter to discontinuation of disruption of said first photo interrupter.

5. The pollinator species detection device of claim 1, wherein detection gates and detection gate arrays are interchangeable where individual gates are sized to accommodate the particular species that is to be observed, tracked, monitored and studied.

6. The pollinator species detection device of claim 1, wherein internal and external sensors can be used to measure real-time internal and external temperature, internal and external humidity, internal and external light intensity, external wind velocity via accelerometers, external rainfall, internal weight and device tilt via inclinometers.

7. The pollinator species detection device of claim 1, wherein the microcontroller and transmittance module can be configured to also receive programming information, via firmware, to modify the content of data collected and transmittance rate from real time to a delayed interval.

8. The pollinator species detection device of claim 1, wherein said power source may be a battery, solar panel, a wind turbine or a combination thereof.

9. The pollinator species detection device in claim 1, wherein a bait may be utilized to attract a specific species and may be an interchangeable and replaceable pheromone, food, honey or sugar that is placed within the confines of said enclosure, externally, or both for emittance and attraction of a specific species.

10. The pollinator species detection device in claim 1, wherein a bait may consist of lights, smells, sounds or pray animals specific to a particular species that are placed in the confines of the enclosure, external to the confines of the enclosure, or both for attraction of a particular species.

11. The pollinator species detection device in claim 1, wherein a sticky paper may be used to enhance, validate or verify sensor detection data.

12. The pollinator species detection device in claim 1, wherein said first sensing, externally residing gates may be fitted with sensors that mimic attractive flowers or fruits attractively specific to a certain species.

13. The pollinator species detection device in claim 1, wherein cameras or microphones may be used internally or externally to capture images and sounds of certain species, respectively.

14. The pollinator species detection device in claim 7, wherein collected data of the number and movement of a species is stored in the device's internal memory and transmitted at programmable and reprogrammable intervals to receivers via radio frequency, Wi-Fi, cellular network or any other suitable form of communication.

15. The pollinator species detection device of claim 14, wherein said pollinator species detection device is capable of adjustable transmission frequencies from real-time to daily, weekly, monthly or yearly, via internalized firmware, adjustable in accord with power source availability and user directed information utilization.

16. The pollinator species detection device of claim 15, wherein stored and transmitted data may be collected in the aggregate across a plurality of devices, areas and regions to create population maps to monitor, track and predict population mechanics and movements.

17. The pollinator species detection device of claim 1, wherein pollinators include winged insects (bees, flies, butterflies, wasps, hornets and moths), winged mammals (birds and bats) or other mammals (primates and rodents).

18. The pollinator species detection device of claim 1, wherein the species studied can be expanded to other non-pollinator species having environmental importance and environmental and inter-species impacts including pests like beetles and locusts.

19. The pollinator species detection device of claim 1, wherein multiple devices may be placed at regular or irregular intervals about a pole or poles, hung in trees or otherwise in places in existing man-made structures at differing heights and positions with different baits to attract different types of pollinators or non-pollinator pests.

20. The pollinator species detection device of claim 1, wherein multiple pollinator species detection devices, each specific to one to a plurality of species, may be placed at irregular and regular intervals in the air, on the ground or under water for species monitoring, data collection and data analysis, singly and in the aggregate.
